(19) United States
(12) Patent Application Publication
(10) Pub. No.: US 2024/0354942 A1
(43) Pub. Date: Oct. 24, 2024

(54) DEEP NEURAL NETWORKS FOR OUTCOME-ORIENTED PREDICTIONS

(71) Applicant: California Institute of Technology, Pasadena, CA (US)

(72) Inventors: Changhuei Yang, South Pasadena, CA (US); Haowen Zhou, Pasadena, CA (US)

(21) Appl. No.: 18/638,327

(22) Filed: Apr. 17, 2024

Related U.S. Application Data
(60) Provisional application No. 63/460,141, filed on Apr. 18, 2023.

(51) Int. Cl. G06T 7/00; G06N 3/045; G16H 50/20

(52) U.S. Cl. CPC G06T 7/0012; G06N 3/045; G16H 50/20; G06T 2207/10056; G06T 2207/30096

(57) ABSTRACT

Among the various aspects of the present disclosure is the provision of deep neural networks for outcome oriented predictions.

### CROSS-REFERENCES TO RELATED APPLICATIONS

This application claims priority to and benefit of U.S. Provisional Patent Application No. 63/460,141, filed on Apr. 18, 2023, which is hereby incorporated by reference in its entirety and for all purposes.

### FEDERALLY SPONSORED RESEARCH OR DEVELOPMENT

This invention was made with government support under Grant No. CA233363 awarded by the National Institutes of Health (NIH). The government has certain rights in the invention.

### FIELD

Certain aspects generally pertain to deep neural networks for outcome-oriented predictions.

### BACKGROUND

Progression of various diseases, such as various types of cancer, are typically predicted by humans, such as pathologists or oncologists. For example, given an incidence of cancer, a pathologist may review biopsy slides and may characterize the tumor as likely to metastasize, or not likely to metastasize. An oncologist may then make treatment determinations based on the likelihood of metastasis. However, predicting disease progression can be difficult and/or inaccurate, which can cause suffering for patients. For example, in an instance in which a tumor is incorrectly predicted as not likely to metastasize, lack of treatment may cause more severe disease, or death. Conversely, in an instance in which a tumor is incorrectly predicted to metastasize, more aggressive treatment may be started, which may produce side effects that could be avoided if it were known prior to aggressive treatment that the tumor is not likely to metastasize.

### SUMMARY

Techniques disclosed herein may be practiced with a processor-implemented method, a system comprising one or more processors and one or more processor-readable media, and/or one or more non-transitory processor-readable media.

In some embodiments, the techniques may involve receiving a microscopy image associated with a test sample. The techniques may further involve identifying a region of interest of the microscopy image for analysis. The techniques may further involve randomly selecting a set of sub-images from within the region of interest. The techniques may further involve generating a set of outcome predictions, each outcome prediction associated with a corresponding sub-image of the set of sub-images by providing the sub-image to a trained deep neural network. The techniques may further involve aggregating the outcome predictions of the set of outcome predictions to generate an aggregate outcome prediction. The techniques may further involve providing the aggregate outcome prediction associated with the microscopy image.

In some embodiments, the techniques may involve obtaining a set of microscopy images and corresponding ground truth predictions, each ground truth prediction indicating an outcome for a patient associated with the microscopy image. The techniques may further involve dividing the set of microscopy images and corresponding ground truth predictions into a training set and a validation set. The techniques may further involve performing an initial training of a deep neural network by: providing sub-images from a region of interest of a given microscopy image from the training set to the deep neural network; generating an aggregate outcome prediction for the given microscopy image based on outcome predictions associated with each sub-image of the given microscopy image; and updating weights of the deep neural network based on a difference between the aggregate outcome prediction and the ground truth prediction for the given microscopy image. The techniques may further involve performing fine-tuning of the deep neural network using the validation set, wherein the fine-tuning comprises updating at least one hyperparameter.

These and other features are described in more detail below with reference to the associated drawings.

### BRIEF DESCRIPTION OF THE DRAWINGS

FIG. 1 is a schematic diagram of components of a system that utilizes deep learning for outcome-oriented predictions in accordance with some embodiments.

FIG. 2 is a flowchart of an example process for generating an outcome-oriented prediction using a trained deep neural network in accordance with some embodiments.

FIG. 3 is a flowchart of an example process for training a deep neural network for generating outcome-oriented predictions.

FIG. 4 illustrates techniques for generating training samples for training a deep neural network for outcome-oriented predictions and an example architecture of a deep neural network in accordance with some embodiments.

FIG. 5 illustrates example training techniques for training a deep neural network in accordance with some embodiments.

FIG. 6 illustrates comparisons 402 of performance of a deep neural network as described herein with performance by pathologists at diagnosing metastatic lung cancer.

FIG. 7 illustrates classifications of various regions of interest by a deep neural network in accordance with some embodiments.

FIG. 8 is a diagram of components of an example computing device in accordance with some embodiments.

These and other features are described in more detail below with reference to the associated drawings.

### DETAILED DESCRIPTION

Different aspects are described below with reference to the accompanying drawings. The features illustrated in the drawings may not be to scale. In the following description, numerous specific details are set forth in order to provide a thorough understanding of the presented embodiments. The disclosed embodiments may be practiced without one or more of these specific details. In other instances, well-known operations have not been described in detail to avoid unnecessarily obscuring the disclosed embodiments. While the disclosed embodiments will be described in conjunction with the specific embodiments, it will be understood that it is not intended to limit the disclosed embodiments.

Progression of various diseases, such as various types of cancer, are typically predicted by humans, such as pathologists or oncologists. For example, given an incidence of cancer, a pathologist may review biopsy slides and may characterize the tumor as likely to metastasize, or not likely to metastasize. An oncologist may then make treatment determinations based on the likelihood of metastasis. However, predicting disease progression can be difficult and/or inaccurate, which can cause suffering for patients. For example, in an instance in which a tumor is incorrectly predicted as not likely to metastasize, lack of treatment may cause more severe disease, or death. Conversely, in an instance in which a tumor is incorrectly predicted to metastasize, more aggressive treatment may be started, which may produce side effects that could be avoided if it were known prior to aggressive treatment that the tumor is not likely to metastasize.

By way of example, non-small cell lung cancer (NSCLC) remains a leading cause of cancer death globally. Despite potentially curative surgery, nearly a third of early-stage (Stage I-III) cases will recur with distant metastases. An increased understanding of tumor biology has suggested that the tumor microenvironment of primary NSCLC may dictate future metastatic behavior. Brain metastases, in particular, are a common cause of morbidity and mortality in NSCLC. Stage of disease is the most commonly used predictor of outcome for NSCLC (and other cancers), but, while stage provides a general risk assessment for a population of patients with similar characteristics, staging is unable to predict which individual patients will or will not progress to metastasis. Histopathologic analysis, even when supplemented by genomic or molecular biomarkers, cannot accurately predict the metastatic potential of NSCLC, particularly in early-stage patients where risk assessment may lead to impactful treatment decisions.

Artificial intelligence has been used to identify subtle features that may predict metastasis. For example, a deep neural network (DNN) may be trained to identify, within a microscopic image provided as input, features that may correlated with tumor metastasis. However, artificial intelligence and neural networks, as have been conventionally implemented thus far, rely on training samples that have been manually annotated (e.g., by a pathologist) to identify regions that correlate with tumor metastasis. The network is then trained based on this manual annotation to identify, in test samples, regions or features of interest that may be correlated with metastasis. However, in cases in which the pathologist is not able to identify regions correlated with disease progression, as in the case of many types of cancer, a neural network trained on pathologist-annotated samples is limited by the performance of the pathologist. For example, in the case of non-small cell lung cancer (NSCLC) progression to brain metastasis, a pathologist may make predictions at a level that is similar to chance, which limits the performance of any network trained using manually-annotated samples.

Disclosed herein are techniques for training a deep neural network (DNN) to generate outcome predictions associated with an input microscopy image. For example, the microscopy image may correspond to a tumor biopsy or other tissue biopsy. The DNN may be configured to generate a prediction of an outcome for the patient associated with the microscopy image. The outcome may correspond to a prediction of disease progression, for example, that a tumor may metastasize from the region of biopsy to a second body region, that death may occur within a given time window, that a cancer may go from early stage to invasive, or the like. Rather than identifying a region of interest in the microscopy image, the DNN may generate a prediction associated with the patient's state or health in the future, e.g., within a given time window. Note that, the DNN may make such a prediction without explicitly identifying regions or features of interest within the microscopy image.

In some embodiments, a region of interest within a microscopy image may be identified. The region of interest may correspond to a tumor or a portion of a tumor, a region in the microenvironment of the tumor, or the like. The region of interest may be sampled to generate a set of sub-images. The number of sub-images may be, e.g., ten, one hundred, one thousand, ten thousand, etc. Note that while a whole slide microscopy image may be on the order of gigapixels, each sub-image may be thousands or tens of thousands of pixels, enabling faster inference time by the DNN. The DNN may generate an outcome prediction for each sub-image, which may be a continuous value, e.g., between −1 and 1, between 0 and 1, etc. The outcome prediction may indicate a likelihood of a particular outcome, such that the tumor will metastasize. The outcome predictions associated with sub-images of the set of sub-images may be aggregated to generate an aggregate outcome prediction. For example, in some embodiments, the aggregate outcome prediction may be a median of the outcome predictions associated with the sub-images. In some embodiments, a threshold may be applied to the aggregate outcome prediction to generate a final classification, e.g., that a tumor will metastasize.

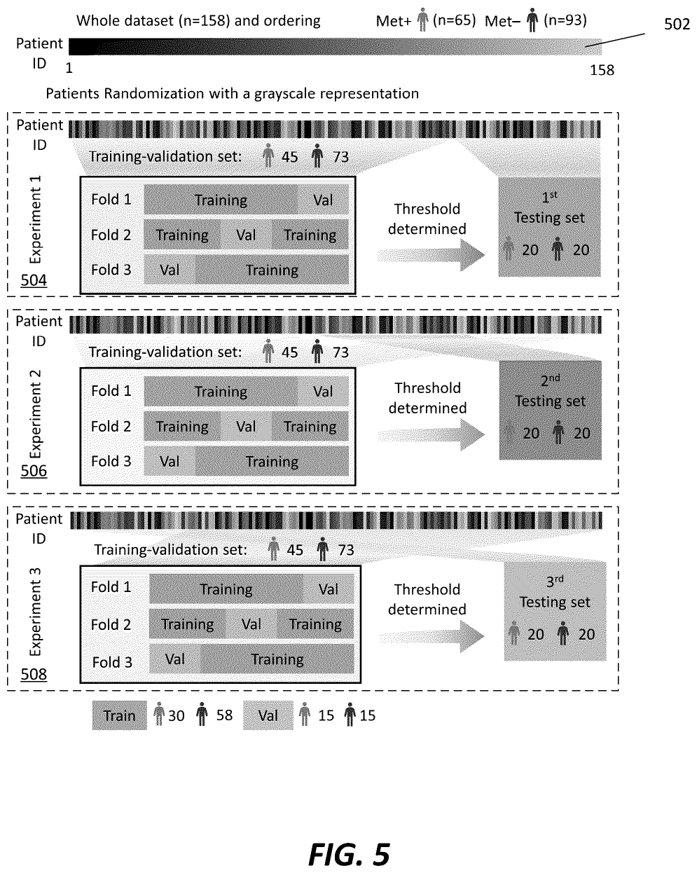

Using the techniques disclosed herein, outcome predictions for disease progression may be substantially improved relative to conventional techniques that involve a pathologist or a machine learning model identifying features of interest in a microscopy image, and subsequently having a pathologist classify progression risk based on the features of interest. As described below in connection with FIGS. 5-7, a DNN trained and utilized using the techniques disclosed herein may have statistically improved performance relative to human pathologists, and may have significantly less false positive and false negative classifications relative to human pathologists. The improved performance of the DNN may lead to improved treatment of patients by correctly providing aggressive treatments for patients at high risk for disease progression, and by avoiding aggressive treatment (which may come with severe side effects) for patients at low risk for disease progression.

It should be noted that the techniques described herein may be performed using microscopy slide images stained with any suitable staining technique (whether conventional physical staining, such as hematoxylin and eosin (H&E) staining, or a virtual staining), or with no staining. Conventionally stained microscopy slides (e.g., stained using H&E staining) may suffer from dye degradation, which may affect how a trained DNN performs. In some cases, microscopy slides may be “virtually stained” using a trained machine learning model. Such a trained machine learning model may utilize generative artificial intelligence (AI) techniques, such as a generative adversarial network (GAN) to generate a virtually stained image corresponding to a raw microscopy image, or a microscopy image that has been digitally processed to determine amplitude and/or phase information. Example techniques for generating virtually stained microscopy images are described in Rivenson, Y., Liu, T., Wei, Z. et al. PhaseStain: the digital staining of label-free quantitative phase microscopy images using deep learning. Light Sci Appl 8, 23 (2019), which is hereby incorporated by reference in its entirety. Separately, conventionally prepared microscopy slides may suffer from lack of uniformity, because the sample may not be homogenously fixed on the slide. This may cause poor focusing, which can affect how a trained DNN may perform. In some embodiments, microscopy images may be digitally focused using computational microscopy, such as Fourier Ptychography (FP). This may allow for post-imaging digital refocusing. Example techniques for performing FP which may be used to digitally refocus microscopy images are described in Zheng, G., Horstmeyer, R. & Yang, C. Wide-field, high-resolution Fourier ptychographic microscopy. Nat. Photonics 7, 739-745 (2013, which is hereby incorporated by reference in its entirety. In other words, as used herein, “microscopy images” which are sampled and/or provided to a DNN as an input image may be conventionally stained microscopy images, virtually stained microscopy images, digitally refocused images, or any combination thereof.

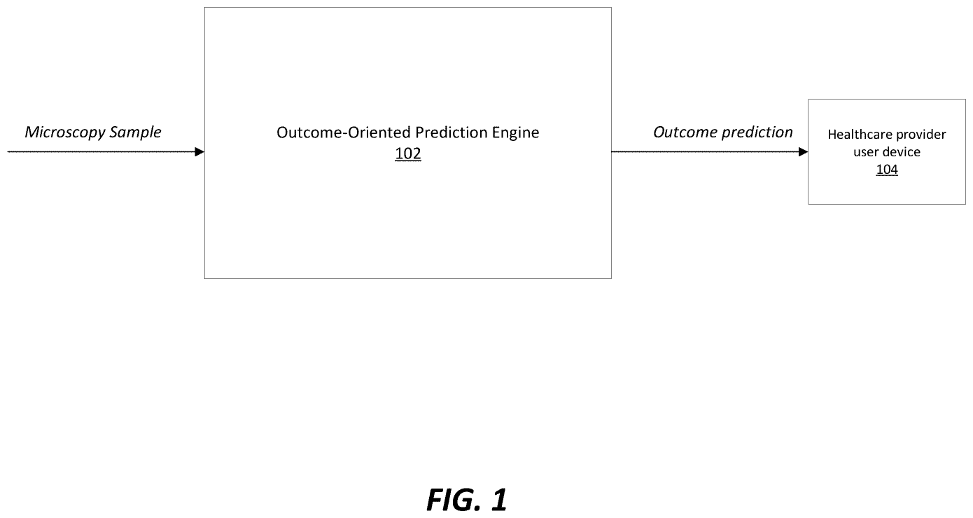

FIG. 1 illustrates an example system that utilizes a deep neural network for outcome-oriented predictions in accordance with some embodiments. As illustrated, an outcome-oriented prediction engine 102 may receive a microscopy sample. The microscopy sample may be represented as an image. The microscopy sample may be generated from a biopsy and subsequent fixation. In some embodiments, the microscopy sample may be stained, e.g., using conventional staining procedures (e.g., H&E staining, or the like). Alternatively, in some embodiments, the microscopy sample may be virtually stained, and/or not stained. In some embodiments, the microscopy sample may include amplitude information and phase information. For example, the microscopy sample may be digitally focused using angular ptychographic imaging with closed-form method (APIC) and may include the amplitude information and the phase information as part of the digital focusing process. Example techniques for utilizing APIC are described in Cao, Ruizhi, Cheng Shen, and Changhuei Yang. “High-resolution, large field-of-view label-free imaging via aberration-corrected, closed-form complex field reconstruction.” arXiv e-prints, pp. arXiv:2309.00755 (2023), which is hereby incorporated by reference in its entirety.

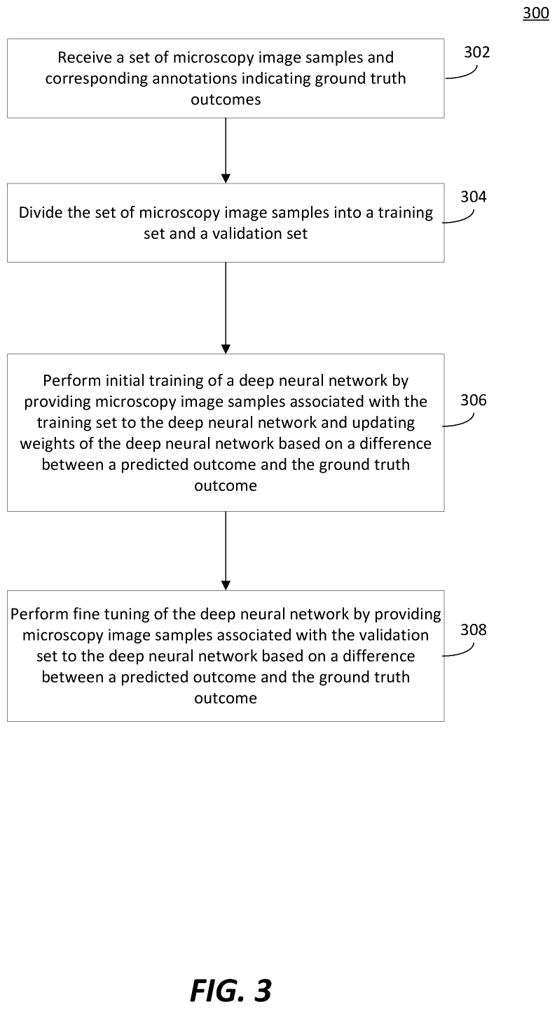

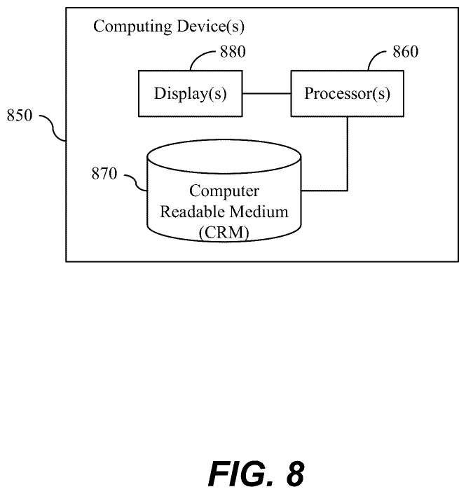

The outcome-oriented prediction engine 102 may include a trained deep neural network. The deep neural network may be utilized at inference time on a server device, a laptop computer, a desktop computer, or any other suitable computing device. Note that, in some embodiments, the deep neural network may be trained on a device (e.g., a server device, a laptop computer, a desktop computer, etc.) that is different from the computing device on which the deep neural network operates at inference time. Example techniques for training a deep neural network are shown in and described below in connection with FIG. 3. Example components of a computing device which may be used to utilize a trained deep neural network at inference time, or to train a deep neural network, are shown in and described below in connection with FIG. 8.

The outcome-oriented prediction engine 102 may take, as input, an image representing the microscopy sample, and generate, as output, an outcome prediction. The outcome prediction may correspond to a likelihood of a progression of a disease state of a patient associated with the microscopy sample progressing. For example, the outcome prediction may correspond to a likelihood of metastasis (e.g., brain metastasis, or the like), a likelihood of death, or any other suitable outcome prediction. Note that the outcome prediction may be associated with a time window. For example, the outcome prediction may represent the likelihood of an event occurring within the time window (e.g., the next five years, the next ten years, etc.). The outcome prediction may be a binary value (e.g., “a high likelihood” versus a “a low likelihood”), or may be a continuous value (e.g., a continuous number from 0 to 1, from −1 to 1, etc.).

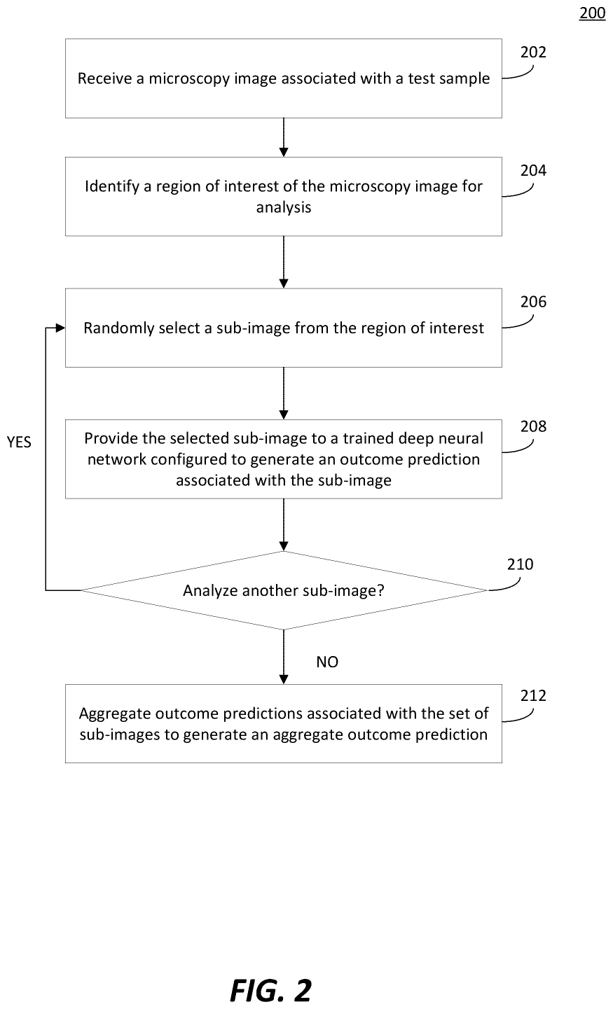

In some embodiments, the DNN that is included in outcome-oriented prediction engine 102 may analyze or consider sub-regions of the microscopy sample image. For example, each sub-region may be randomly selected. As a more particular example, each sub-region may be randomly selected from a region corresponding to a tumor region. In some embodiments, the DNN may assign an outcome prediction to each sub-region. The outcome-oriented prediction engine 102 may then aggregate the outcome predictions associated with each sub-region. Example techniques for utilizing a DNN to make outcome predictions are shown in and described below in connection with FIG. 2.

In some embodiments, the outcome prediction may be provided to a healthcare user device 104. Healthcare user device 104 may be a mobile phone, a laptop computer, a tablet computer, a desktop computer, a server device, a cloud service device, etc. For example, in some embodiments, healthcare user device 104 may be a mobile device of a physician, such as an oncologist. The physician may then be able to use the outcome prediction to tailor treatment to the patient based on the prediction. As another example, in some embodiments, healthcare user device 104 may be a server or other remote device, e.g., associated with an electronic healthcare records system or the like. In some embodiments, a physician may be able to access the server or remote device, e.g., via a desktop computer, laptop computer, or the like to access the outcome prediction and thereby determine suitable treatment options based on the outcome prediction.

In some embodiments, a trained DNN may be used to generate outcome predictions associated with a test sample. For example, a DNN may receive a microscopy image associated with the test sample, or may receive a sub-image from the microscopy image. The image, or the sub-image, may then be provided to the DNN as an input. The DNN may generate an output that corresponds to an outcome prediction. For example, the outcome prediction may be a prediction of whether or not a particular disease progression (e.g., metastasis of a tumor, death, etc.) will occur within a future time window (e.g., within the next year, within the next five years, within the next ten years, etc.). In some embodiments, the DNN may additionally generate a confidence associated with the outcome prediction. In some embodiments, the outcome prediction may be a continuous value (e.g., between −1 and 1, between 0 and 1, etc.) representing a probability of a particular outcome occurring (e.g., that metastasis will occur, or the like). In some embodiments, the outcome prediction may be a binary value corresponding to “yes” the outcome is likely, or “no” the outcome is not likely. In instances in which a binary value is generated, the binary value may be generated by comparing a probability to a threshold and determining the binary value based on the comparison of a probability of the outcome occurring to the threshold.

In some embodiments, a DNN may consider multiple sub-images of a microscopy image. For example, the DNN may consider ten, fifty, one hundred, one thousand, ten thousand, etc. sub-images of a microscopy image. In some embodiments, the DNN may generate an outcome prediction and/or a confidence level for each sub-region. The outcome predictions associated with each sub-region analyzed may then be aggregated to generate an aggregate outcome prediction. For example, in an instance in which the outcome prediction is a continuous value (e.g., from −1 to 1, 0 to 1, or the like), the median outcome prediction may be determined from the outcome predictions of all of the sub-regions to determine the aggregate outcome prediction. In some embodiments, each sub-region may be equally weighted to determine the aggregate outcome prediction. Alternatively, in some embodiments, different sub-regions (e.g., those near a center of a tumor region, those near an edge of a tumor region, etc.) may be weighted more or less heavily than other sub-regions.

FIG. 2 is a flowchart of an example process 200 for generating outcome predictions using a DNN in accordance with some embodiments. Note that blocks of process 200 may be performed using a trained DNN. Example techniques for training such a DNN are shown in and described below in connection with FIG. 3. In some embodiments, blocks of process 200 may be executed by a computing device, such as a server device, a laptop computer, a desktop computer, or the like. Example components of such a computing device are shown in and described below in connection with FIG. 8. In some embodiments, blocks of process 200 may be executed in an order other than what is shown in FIG. 2. In some embodiments, two or more blocks of process 200 may be executed substantially in parallel. In some embodiments, one or more blocks of process 200 may be omitted.

Process 200 can begin at 202 by receiving a microscopy image associated with a test sample. The test sample may be, e.g., a biopsy of a tumor or other tissue. The test sample may be fixed to form a whole slide. The microscopy image may be a physically stained image (e.g., using H&E staining), virtually stained using one or more machine learning models (e.g., machine learning models distinct from the DNN), or not stained. The microscopy image may be one that is digitally focused. The microscopy image may contain amplitude and phase information.

At 204, process 200 can identify a region of interest of the microscopy image for analysis. In some embodiments, the region of interest may correspond to a region or a boundary of a particular type of tissue, such as a tumor. In some embodiments, the region of interest may be identified based on manual annotation of the microscopy image. For example, a tumor region may be identified by a pathologist. As another example, in some embodiments, the region of interest may be identified by a trained neural network (e.g., a convolutional neural network) trained to identify boundaries of particular content, such as a tumor region. In some embodiments, process 200 can generate a mask based on the region of interest. For example, in some embodiments, process 200 can perform thresholding to generate a mask with binary pixel values, with pixel values corresponding to either within the region of interest or not within the region of interest.

At 206, process 200 can randomly select a sub-image from the region of interest. For example, in some embodiments, process 200 can select a set of pixels that correspond to the sub-image, where the set of pixels are within the region of interest. In some embodiments, the sub-image may be selected with uniform probability from within the region of interest. Alternatively, in some embodiments, the sub-image may be selected with non-uniform probability from within the region of interest, such that regions near an edge of the region of interest, or regions within a center of the region of interest are more likely to be selected.

At 208, process 200 can provide the selected sub-image to a trained DNN configured to generate an outcome prediction associated with the sub-image. As described above, the outcome prediction may indicate a likelihood of progression of a disease state of a patient associated with the test sample. For example, in an instance in which the microscopy image depicts a portion of a tumor in a first body region (e.g., the lung of the patient), the outcome prediction may indicate metastasis to a second body region (e.g., the brain). The outcome prediction may be a continuous value (e.g., between 0 and 1, between −1 and 1, or the like). Alternatively, the outcome prediction may be a binary value. In some embodiments, the outcome prediction may be associated with a confidence level, where lower levels of confidence indicate less accuracy or confidence in the outcome prediction.

It should be noted that the input layer of the DNN may be configured based on the type of microscopy image that is obtained. For example, the DNN may include an input layer configured to accept three color channels of information in instances in which the microscopy image is physically stained. In instances in which the microscopy image is not stained, the DNN input layer may be configured to accept a single channel of phase information or two channels with amplitude and phase information. In instances in which the microscopy image is virtually stained, the DNN input layer may be configured to accept one channel or two channels of information, which may include absorption information in an amplitude channel.

At 210, process 200 can determine whether another sub-image is to be analyzed. In some embodiments, process 200 may analyze a predetermined number of sub-images (e.g., ten, fifty, one hundred, one thousand, ten thousand, etc.) from within the region of interest. In some embodiments, process 200 may determine another sub-image is to be analyzed responsive to determining that the number of sub-images that have thus far been analyzed is less than the predetermined number. In some embodiments, process 200 may determine whether another sub-image is to be analyzed based on a variance in the outcome predictions for sub-regions that have been thus far analyzed. For example, in an instance in which the outcome predictions have high variance (e.g., a variance that is greater than a predetermined threshold), indicating high variability across the different sub-regions, process 200 may determine that additional sub-images are to be analyzed. Conversely, in an instance in which the outcome predictions have low variance indicating low variability in the outcome predictions associated with sub-images across the region of interest, process 200 may determine that no additional sub-images are needed.

If, at 210, process 200 determines that additional sub-images are to be analyzed (“yes” at 210), process 200 can loop back to 206 and can randomly select another sub-image from the region of interest. Process 200 can loop through blocks 206-210 until process 200 determines that no additional sub-images are to be analyzed.

Conversely, if, at 210, process 200 determines that no additional sub-images are to be analyzed (“no” at 210), process 200 can proceed to block 212 and can aggregate outcome predictions associated with the set of sub-images to generate an aggregate outcome prediction. For example, in some embodiments, the aggregate outcome prediction can be a median of the outcome predictions associated with the set of sub-images, a mean of the outcome predictions associated with the set of sub-images, a weighted average of outcome predictions associated with the set of sub-images, or the like. In some embodiments, one or more outlier outcome predictions may be discarded prior to determining the aggregate outcome prediction. For example, outlier outcome predictions may be identified using a clustering technique (e.g., to identify outcome predictions outside one or more clusters), based on outcome prediction values that are more than a threshold number of standard deviations away from a mean outcome prediction, or the like.

The aggregate outcome prediction may be a continuous value that represents a probability of disease progression, e.g., a value from −1 to 1 or from 0 to 1. In some embodiments, the aggregate outcome prediction may be transformed to a binary value. For example, a probability of disease progression may be compared to a threshold (e.g., a probability of 0.5, a probability of 0.6, etc.), and aggregate outcome predictions that exceed the threshold may be classified as “likely progression,” and aggregate outcome predictions below the threshold may be classified as “unlikely to progress.” Note that, in some embodiments, the threshold may be determined as part of the training of the DNN, e.g., during a validation phase of the DNN. For example, the threshold may be selected during the validation phase to achieve particular sensitivity and/or selectivity metrics with a training set and a validation set.

In some embodiments, a DNN may be trained using a training set and a validation set. In some embodiments, the training set may be used during an initial training phase to determine weights associated with the DNN. In some embodiments, after the initial training phase, the validation set may be used to tune hyperparameters of the DNN. The hyperparameters may include learning rate, batch size, weight decay, number of epochs, and/or learning scheduler. In some embodiments, a group of manually annotated microscopy images may form a combined training set and validation set. Each microscopy image may be manually annotated for regions of interest associated with a ground truth outcome. For example, in the case of microscopy images that correspond to tumor biopsy images, the ground truth outcome may indicate a disease progression outcome, such as whether metastasis to a different body region occurred. For example, for a given microscopy image, a corresponding label may be Met+ (indicating that metastasis occurred), or Met− (indicating metastasis did not occur). Note that other outcomes may be used rather than metastasis, such as death of the patient, a cancer progressing from early stage to invasive, etc. The training set and the validation set may be constructed such that each of the training set and the validation set include a balance of samples associated with each ground truth outcome. For example, the training set may include 20 Met+ and 20 Met− training samples, 30 Met+ and 50 Met− training samples, or any other suitable number. Similarly, the validation set may include a mix of samples associated with each ground truth outcome.

Each training sample (during the initial training phase) or each validation sample (during the fine tuning stage) may be provided to the DNN in multiple instances. For example, a microscopy image may be randomly sampled a number of times to generate a corresponding number of sub-regions that are each provided to the DNN. As described above in connection with FIG. 2, an outcome prediction may be generated for each sub-region. The outcome predictions for each sub-region may be aggregated (e.g., using a mean, a median, a weighted average, etc.) to determine an aggregate outcome prediction. The aggregate outcome prediction, which may be a continuous value (e.g., between −1 and 1, between 0 and 1, etc.) representing probability of a given outcome may be compared to a threshold to determine a binary aggregate outcome prediction. During the initial training phase, weights of the DNN may be updated based on a comparison of the binary aggregate outcome prediction to the ground truth outcome prediction. During the validation phase, hyperparameters may be tuned or modified based on a comparison of the binary aggregate outcome to the ground truth outcome. Note that, prior to providing a sub-region image to the DNN (whether in the initial training phase or the validation phase), color normalization or any other suitable pre-processing may be performed. Additionally, it should be noted that images used to train the DNN may be physically stained, virtually stained (e.g., using a machine learning model other than the outcome-prediction DNN), or not stained. However, the DNN is trained using the same type of staining as is used by the DNN at inference time.

FIG. 3 is a flowchart of an example process 300 for training a DNN in accordance with some embodiments. In some embodiments, blocks of process 300 may be executed by a computing device, such as a server device, a laptop computer, a desktop computer, or the like. Example components of such a computing device are shown in and described below in connection with FIG. 8. In some embodiments, blocks of process 300 may be executed in an order other than what is shown in FIG. 3. In some embodiments, two or more blocks of process 300 may be executed substantially in parallel. In some embodiments, one or more blocks of process 300 may be omitted.

Process 300 can begin at 302 by receiving a set of microscopy image samples and corresponding annotations indicating ground truth outcomes. For example, each microscopy image sample may be a whole slide image (WSI) representing, e.g., a tumor biopsy or other sample. Each image sample may be associated with a ground truth outcome, which may correspond to a ground truth state of disease progression (e.g., metastasis occurred, metastasis did not occur, etc.) within a given time window (e.g., one year, five years, ten years, etc.). Ground truth outcomes may be obtained from manual annotations (e.g., by a physician), and/or obtained from a health records database.

At 304, process 300 can divide the set of microscopy image samples into a training set and a validation set. Each of the training set and the validation set may include a mix of ground truth outcomes. For example, in an instance in which the ground truth outcomes include whether or not a tumor metastasized (e.g., Met+ and Met−), the training set may include a mix of Met+ and Met− samples, and similarly, the validation set may also include a mix of Met+ and Met− samples. Note that the number of each outcome may be the same for the training set and/or the validation set (e.g., 30 Met+ and 30 Met−, 50 Met+ and 50 Met−), or may be different (e.g., 30 Met+ and 60 Met−, 60 Met+ and 40 Met−), or the like. Note that samples included in the training set are not included in the validation set, and vice versa.

At 306, process 300 can perform initial training of the DNN by providing microscopy image samples associated with the training set to the DNN and updating weights of the DNN based on a difference between a predicted outcome and the ground truth outcome. For example, in some embodiments, process 300 can generate a set of sub-region images from a given microscopy image sample by sampling the microscopy image sample (e.g., ten samples, one hundred samples, one thousand samples, ten thousand samples, etc.). Continuing with this example, process 300 can provide each sub-region image to the DNN to generate an outcome prediction. Process 300 can then generate an aggregate outcome prediction by aggregating the outcome predictions associated with each sub-region (e.g., by determining a mean, a median, a weighted average, etc.). The aggregate outcome prediction, which may be a continuous value, may then be transformed to a binary value, e.g., by comparing the continuous value to a threshold. The weights may then be updated based on a comparison to the ground truth outcome. This process may be repeated for each microscopy image in the training set. Note that weights of the DNN may be updated in batches, each batch comprising multiple training sample instances. Weights may be updated using any suitable techniques, such as backpropagation using gradient descent, etc.

At 308, process 300 can perform fine tuning of the DNN by providing microscopy image samples associated with the validation set to the DNN. During the fine tuning stage, hyperparameters of the DNN (e.g., learning rate, batch size, weight decay, number of epochs, and/or learning scheduler) may be updated based on a difference between a predicted outcome associated with a validation set sample and the ground truth outcome. Note that, as used herein, a “learning scheduler” may be a process that allows control of how the learning rate is modified, e.g., according to learning schedule hyperparameter, or based on performance improvements (which may be specified as one or more hyperparameters). Similar to what is described above in connection with block 306, during the fine tuning stage, process 300 may sample a microscopy image sample of the validation set to generate a set of sub-region images, each of which are provided to the DNN. An outcome prediction may be generated by the DNN for each sub-region image, which may then be aggregated to form an aggregate outcome prediction. The aggregate outcome prediction may be transformed to a binary outcome prediction, and the hyperparameters may be updated based on a comparison of the binary outcome prediction to the ground truth outcome prediction.

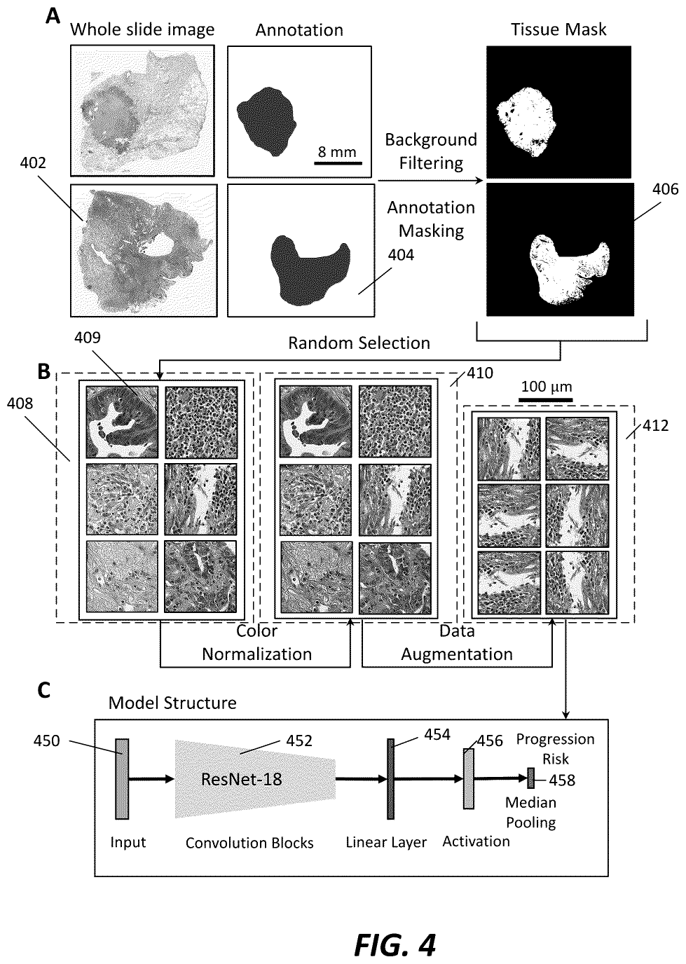

FIG. 4 illustrates example techniques for generating samples (e.g., training samples, validation samples, and/or test samples) provided as input to a DNN as well as an example DNN architecture that may be utilized.

As shown in panel A of FIG. 4, a whole slide image (such as whole slide image 402) is obtained, which may depict a test sample. The test sample may include a tumor biopsy, a biopsy of other tissue, or any suitable type of test sample. The whole slide image is a microscopy image. In the example shown in FIG. 4, the microscopy image is physically stained (e.g., using H&E staining), however, it should be understood that in some embodiments, the microscopy image may be virtually stained, or may not be stained at all. In some embodiments, the microscopy image may be digitally focused.

An annotation of a region of interest, such as annotation 404, may be used to generate a mask, such as mask 406. For example, annotation 404 indicates a tumor region present in whole slide image 402. Based on the annotation, background regions not part of the annotated region of interest may be filtered out to generate the mask. The annotation may be obtained from manual annotation (e.g., a pathologist may indicate regions of the whole slide image that indicate a region of interest). Additionally or alternatively, in some embodiments, the annotation may be generated using a trained machine learning model, such as a trained convolutional network, which may be trained to identify bounds of a region of interest. Such a trained machine learning model may be separate from a DNN configured to perform outcome prediction, and may itself be trained using manually annotated samples.

For each whole slide image, a set of sub-images from within the region of interest may be generated, where each sub-image is provided separately as input to the DNN. For example, set of sub-images 408 include random samples from a region of interest identified in mask 406. For example, sub-image 409 is included in set of sub-images 408. Each sub-image may be of any suitable size, such as 100 pixels by 100 pixels, 150 pixels by 150 pixels, 300 pixels by 300 pixels, 400 pixels by 400 pixels, etc. Note that each sub-image may be obtained by random sampling within the region of interest. Additionally, note that while the whole slide image may have a size in the range of megapixels or even gigapixels, each sub-image may be substantially smaller, e.g., on the order of thousands or tens of thousands of pixels.

Each sub-image may undergo pre-processing, such as color normalization. For example, color normalized set of sub-images 410 correspond to set of sub-images 408 after color normalization has been performed to standardize the set of sub-images. Note that, in instances in which microscopy images are either virtually stained or are not stained, color normalization may be omitted.

Samples included in a training set may undergo a data augmentation procedure to expand the range of samples a DNN is trained on. For example, as shown in set of sub-images 412, data augmentation may involve random cropping, flipping, rotating, etc. sub-images from set of sub-images 408 and/or 410.

Panel C of FIG. 4 depicts an example architecture of a DNN that may be used in accordance with some embodiments. As illustrated, at input 450 is provided as an input to a set of convolutional blocks 452. Input 450 may be a vector representation of a sub-image. In an initial training phase, the sub-image may be from a training set (e.g., a sub-image sampled from a whole slide image assigned to a training set, or a sub-image obtained from data augmentation from a sub-image sampled from a training set whole slide image). In a fine tuning stage, the sub-image may be from a validation set (e.g., a sub-image sampled from a whole slide image assigned to a validation set, or a sub-image obtained from data augmentation from a sub-image sampled from a training set whole slide image). At an inference state, the sub-image may be from a test set (e.g., a sub-image sampled from a test whole slide image).

Convolution blocks 452 may comprise one or more convolutional layers. Each convolutional layer may be configured to extract features of input 450. Extracted features may be passed to a subsequent convolutional layer. In the example shown in FIG. 4, a ResNet-18 architecture with 18 convolutional layers is used, but this is merely an example, and any suitable number of convolutional blocks or layers may be used (e.g., five, ten, twenty, one hundred, etc.).

The output of convolutional blocks 452 is provided to a linear layer 454. A linear layer, sometimes referred to as a fully-connected layer, connects every input node to every output node. The output of linear layer 454 is provided to an activation function 456. In some embodiments, activation function 456 may be a sigmoid function, or a softmax function. The output of activation function 456 may be a continuous value (e.g., between −1 and 1, between 0 and 1, etc.) that indicates a likelihood of a given outcome (e.g., metastasis, transition of a cancer from early stage to invasive, etc.).

Note that, as described above in connection with FIGS. 2 and 3, multiple sub-images may be provided to a DNN, with output predictions generated for each sub-image. The output predictions for the set of sub-images may then be aggregated using median pooling layer 458. For example, the output predictions from each sub-image may be aggregated and a median output prediction may be identified using median pooling layer 458. The output of median pooling layer 458 may correspond to the aggregate output prediction. As described above, aggregating output predictions by finding a median output prediction is merely one example, and, in some embodiments, output predictions may be aggregated using a mean (with or without outlier data discarded), a weighted average, or the like.

In some embodiments, a DNN may be pre-trained using an image database prior to an initial training phase that utilizes a training set of microscopy images. The weights obtained using the pre-training phase may be used to initialize the DNN at the start of the initial training phase.

It should be noted that a DNN may have any suitable architecture and is not limited to the architecture shown in panel C of FIG. 4. For example, in some embodiments, a DNN may have one or more attention layers or other attention mechanisms, such as those implemented in a transformer network. As another example, in some embodiments, a DNN may use different activation functions, such as sigmoid, softmax, etc. As yet another example, in some embodiments, a DNN may execute different pooling functions, such as attention pooling, average pooling, etc. Such attention mechanisms may allow a DNN to select regions that should be attended to when determining predictions.

### Experimental Data

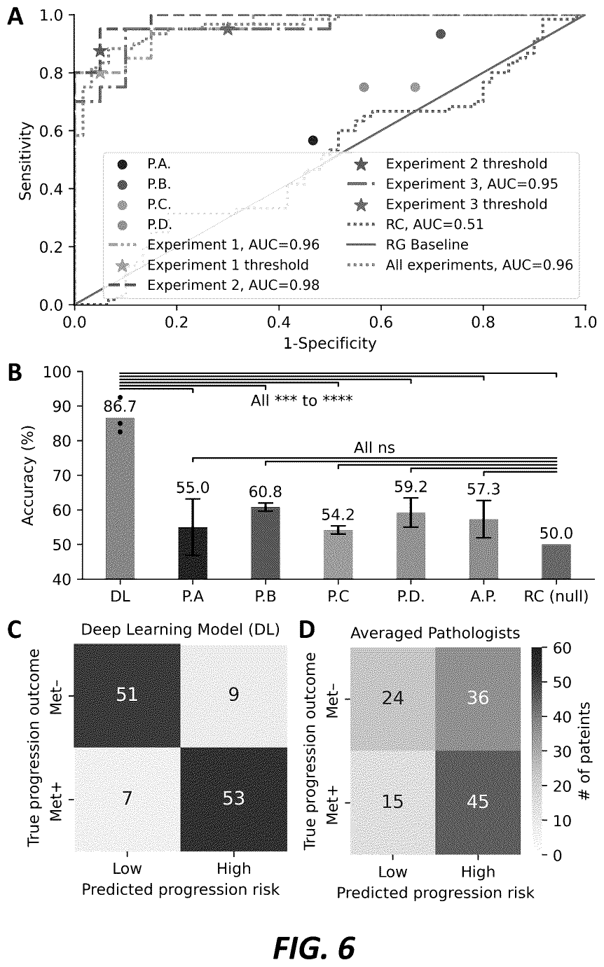

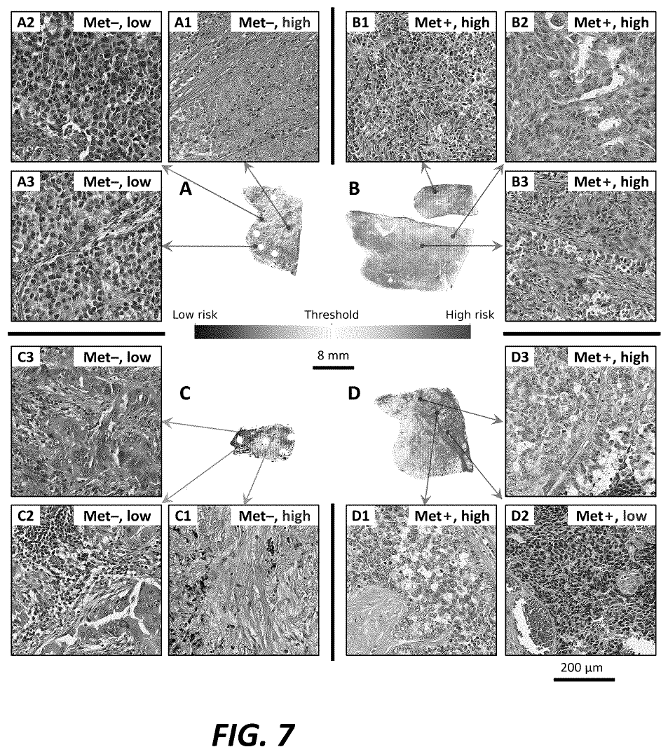

FIG. 5 illustrates techniques associated with an experiment conducted using a trained DNN and physically stained microscopy images, and FIGS. 6 and 7 illustrate experimental results obtained using the trained DNN.

Turning to FIG. 5, a whole dataset 502 included microscopy images from 158 patients with NSCLC. Of those 158, 65 had progression to brain metastasis within five years (designated as Met+), and 93 did not have progression to brain metastasis within five years (designated as Met−).

Three experiments were conducted using whole dataset 502, depicted in panels 504, 506, and 508, respectively. The whole dataset 502 was randomized, and the randomized sequence was used to divide the patient population into a training/validation set and a testing set for each experiment. The training/validation set included 118 patients, 45 Met+ and 73 Met−, and the testing set included 40 patients, 20 Met+ and 20 Met− in each experiment. Note that, although the numbers of total patients and the split between Met+ and Met− patients was the same for each set across the different experiments, different patient subsets were chosen for each experiment. The patients in the training and validation sets were at least partially overlapping, while the patients in the testing set for each experiment were entirely different.

Within each experiment, three rounds of cross-validation training were performed, depicted as “Fold 1,” “Fold 2,” and “Fold 3” in FIG. 5. In each round, the DNN was trained on 88,000 image tiles derived from 88 whole slide images in the training set (e.g., 1000 sub-images were sampled from each whole slide image). In each round, the model was validated on 30,000 image tiles derived from 30 whole slide images in the validation set. In the training and validation rounds, ground truth progression outcomes (e.g., Met+ or Met−) were used as labels to update weights and/or hyperparameters. The training/validation process was iterated three times, using a different set of 88 cases for training and a different 30 cases for validation. During the process, validation accuracy was optimized by altering the model hyperparameters (e.g., learning rate, batch size, weight decay, number of epochs, and/or learning scheduler), and the model was then retrained on the entire set using the optimized parameters. After the three rounds, in each experiment, the model was tested using the 40 cases assigned to the test set. Thresholds for converting an outcome prediction (e.g., a value from −1 to 1 or a value from 0 to 1) to a binary outcome were determined during the validation process. The procedure was repeated for each of the three experiments, and the results of the three experiments are shown in and described below in connection with FIG. 6.

To assess the effectiveness of the DNN trained in accordance with the techniques shown in FIG. 5 in predicting progression risk, the receiver operating characteristic (ROC) curve and the area under the ROC curve (AUC) were calculated to provide a measure of the performance of the model. To compare the model outputs with clinical progression outcomes, the model prediction scores were binarized to generate an accuracy metric. P-values were calculated to show the performances of the model compared to pathologists. Performance was also compared to performance of a random classifier assuming the null-hypothesis of the random classifier.

The predictive performance of a DL-based classifier was evaluated in three rounds using non-overlapping (e.g., distinct) patients in the testing set for each round, as described above in connection with FIG. 5. The resulting AUC after each training and validation session that utilized different cases was 0.96, 0.98, and 0.95, as shown in panel A of FIG. 6, with optimal sensitivities of 80%, 90%, and 95%, and specificities of 90%, 95%, 70%, respectively. In contrast, a similar training and validation session that employed a random assignment of progression phenotype labels (e.g., a random classifier) yielded an AUC of 0.51, as shown in panel A of FIG. 6.

When the optimal cut-off was chosen from each training and validation session and applied to the separate set of test cases, an average accuracy of 87% was achieved, as shown in panel B of FIG. 6 (p<0.00001, compared to accuracy based on random classifier training).

Although there are no reliable histopathologic features that can be routinely used to predict metastatic risk progression in NSCLC, the ability of four independent expert lung pathologists to provide a similar binary diagnosis from the same test set used by the DNN was tested. Results of the pathologists are labeled “P.A,” “P.B,” “P.C.,” and “P.D.” in panels A and B of FIG. 6. Pathologists were provided with whole slide images, divided into the same three testing sets as implemented for the DNN testing. Compared to the trained DNN model, the average accuracy across the three test sets among four pathologists was 55.0%, 60.8%, 54.2%, and 59.2% respectively (as shown in panel B of FIG. 6). The average accuracy of the pathologists was 57.3%, which is not significantly different compared to prediction accuracy based on random classifier training (p>0.05). The individual sensitivity and specificity of prediction from the four pathologists were 57%, 93%, 75%, 75% and 53%, 28%, 33% 43%, respectively, suggesting that the DNN identified features that were not readily discernable by a trained pathologist (e.g., tumor grade, necrosis, lymphocytic infiltration, spread to airway spaces), and that the DNN model outperformed careful histologic review by experienced pathologists (p<0.001).

Panel C of FIG. 6 illustrates the true positive, true negative, false positive, and false negative predictions of the DNN relative to the average of the pathologists. Note that, for all categories, the DNN outperformed the pathologists. For example, while the DNN had 7 instances of a false negative prediction (e.g., that the tumor metastasized while the DNN predicted no metastasis), on the same test set, the pathologists on average had 15 false negative predictions. A false negative prediction may lead to lack of aggressive treatment for patients whose tumor metastasizes, which could lead to earlier death and increased suffering. Conversely, the DNN had 9 instances of a false positive (e.g., a prediction that the tumor will metastasize, where no such metastasis occurred), whereas, on the same test set, the pathologists had 36 false positive instances. An inaccurate false positive may lead to aggressive treatment that is unwarranted, which may lead to severe side effects and suffering by patients. Accordingly, the improved performance of the DNN relative to conventional prediction by human pathologists may lead to substantially improved treatment and less suffering (whether due to accurate treatment of tumors likely to metastasize, or due to side effects from unnecessary aggressive treatment) for patients.

As described above, the improved performance of the DNN relative to that of pathologists suggests that the DNN is capable of identifying features of the microscopy image that are too subtle to be detected by pathologists. Results of an investigation of the DNN's attention at the sub-image (or tile) level for a given whole slide image is shown in FIG. 7. As described above, the DNN was trained and tested on 1,000 sub-images sampled from a region of interest and the immediately surrounding tumor environment to generate an outcome prediction for each sub-image, and the individual outcome predictions were aggregated to generate an aggregate prediction. FIG. 7 illustrates several examples of prediction/attention maps showing the areas that the DNN determined as significant for determining an outcome for each case. Note that each example shown in FIG. 7 is associated with a correct prediction (e.g., Met+ or Met−) for the given whole slide image made by the DNN.

As can be seen in FIG. 7, the tumor regions identified by the DNN as high-risk or low-risk had little in the way of discernable histologic differences, and both high-risk and low-risk regions as identified by the DNN showed tumor cells, tumor microenvironment, and acellular stromal components. This suggests that the DNN training considered a broad and perhaps unappreciated set of histological features and not (or at least not only) the characteristic nuclear or cellular characteristics that are conventionally used in tumor grading methods. Moreover, as can be seen in the examples depicted in FIG. 7, cases correctly identified by the DNN as Met+ or Met− did not necessarily demonstrate uniform scores across the sub-images. For example, many cases corrected classified as Met+ included sub-images that were individually classified as low risk, but in aggregate, the DNN identified sufficient sub-images that were classified as high risk to, in aggregate, generate a Met+ classification. The heterogeneity of the DNN's predictions for each sub-image may reflect tumor heterogeneity with respect to molecular phenotype and metastatic potential.

### Computational Systems

The techniques described above may be implemented using one or more computing devices. For example, a DNN may be trained and/or utilized at inference time using a computational device such as a server device, a laptop computer, a desktop computer, or the like. FIG. 8 illustrates an example computing device that may be used, e.g., to implement blocks of process 200 and/or 300 of FIGS. 2 and/or 3, respectively.

In FIG. 8, the computing device(s) 850 includes one or more processors 860 (e.g., microprocessors), a non-transitory computer readable medium (CRM) 870 in communication with the processor(s) 860, and one or more displays 880 also in communication with processor(s) 860.

Processor(s) 860 is in electronic communication with CRM 870 (e.g., memory). Processor(s) 860 is also in electronic communication with display(s) 880, e.g., to display image data, text, etc. on display 880.

Processor(s) 860 may retrieve and execute instructions stored on the CRM 870 to perform one or more functions described above. For example, processor(s) 860 may execute instructions to perform one or more operations to generate an outcome prediction, train a DNN to generate outcome predictions, etc.

The CRM (e.g., memory) 870 can store instructions for performing one or more functions of the described above. These instructions may be executable by processor(s) 870. CRM 870 can also store raw images, e.g., microscopy images, sub-images sampled from an image, or the like.

Modifications, additions, or omissions may be made to any of the above-described embodiments without departing from the scope of the disclosure. Any of the embodiments described above may include more, fewer, or other features without departing from the scope of the disclosure. Additionally, the steps of described features may be performed in any suitable order without departing from the scope of the disclosure. Also, one or more features from any embodiment may be combined with one or more features of any other embodiment without departing from the scope of the disclosure. The components of any embodiment may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C#, C++ or Python, Matlab, or other suitable language/computational software, including low level code, including code written for field programmable gate arrays, for example in VHDL; embedded artificial intelligence computing platform, for example in Jetson. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random-access memory (RAM), a read only memory (ROM), a magnetic media such as a hard-drive or a floppy disk, or an optical media such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed embodiments have been described in some detail to facilitate understanding, the described embodiments are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

### Example Embodiments

Embodiment 1: A method comprising: receiving a microscopy image associated with a test sample; identifying a region of interest of the microscopy image for analysis; randomly selecting a set of sub-images from within the region of interest; generating a set of outcome predictions, each outcome prediction associated with a corresponding sub-image of the set of sub-images by providing the sub-image to a trained deep neural network; aggregating the outcome predictions of the set of outcome predictions to generate an aggregate outcome prediction; and providing the aggregate outcome prediction associated with the microscopy image.

Embodiment 2: The method of embodiment 1, wherein an outcome prediction of the set of outcome predictions corresponds to a prediction of disease progression within a future time period.

Embodiment 3: The method of embodiment 2, wherein the prediction of disease progression comprises metastasis of a tumor to a body region different from a body region associated with the test sample.

Embodiment 4: The method of any one of embodiments 1-3, wherein the microscopy image is a microscopy image that has not been physically stained.

Embodiment 5: The method of embodiment 4, wherein the microscopy image comprises a virtually stained microscopy image.

Embodiment 6: The method of embodiment 5, wherein the virtually stained microscopy image was generated by a trained machine learning model different from the trained deep neural network.

Embodiment 7: The method of any one of embodiments 1-6, wherein identifying the region of interest comprises filtering a background region based on an annotation of the region of interest.

Embodiment 8: The method of any one of embodiments 1-7, wherein the set of sub-images are selected with uniform probability from within the region of interest.

Embodiment 9: The method of any one of embodiments 1-8, wherein the deep neural network comprises a convolutional neural network.

Embodiment 10: The method of any one of embodiments 1-9, wherein the deep neural network comprises an attention mechanism configured to utilize a region of interest within a sub-image to generate a corresponding outcome prediction.

Embodiment 11: The method of any one of embodiments 1-10, wherein the aggregate outcome prediction is a median outcome prediction of the set of outcome predictions.

Embodiment 12: A method comprising: obtaining a set of microscopy images and corresponding ground truth predictions, each ground truth prediction indicating an outcome for a patient associated with the microscopy image; dividing the set of microscopy images and corresponding ground truth predictions into a training set and a validation set; performing an initial training of a deep neural network by: providing sub-images from a region of interest of a given microscopy image from the training set to the deep neural network; generating an aggregate outcome prediction for the given microscopy image based on outcome predictions associated with each sub-image of the given microscopy image; and updating weights of the deep neural network based on a difference between the aggregate outcome prediction and the ground truth prediction for the given microscopy image; and performing fine-tuning of the deep neural network using the validation set, wherein the fine-tuning comprises updating at least one hyperparameter.

Embodiment 13: The method of embodiment 12, wherein the at least one hyperparameter comprises a learning rate, a batch size, a weight decay, a learning scheduler, or any combination thereof.

Embodiment 14: The method of any one of embodiments 12-13, wherein the fine-tuning of the deep neural network comprises providing sub-images from microscopy images included in the validation set to the initially-trained deep neural network.

Embodiment 15: The method of any one of embodiments 12-14, wherein the microscopy image is a microscopy image that has not been physically stained.

Embodiment 16: The method of embodiment 15, wherein the microscopy image comprises a virtually stained microscopy image.

Embodiment 17: A system comprising: one or more processors; and one or more processor-readable media storing instructions which, when executed by one or more processors, cause performance of: receiving a microscopy image associated with a test sample; identifying a region of interest of the microscopy image for analysis; randomly selecting a set of sub-images from within the region of interest; generating a set of outcome predictions, each outcome prediction associated with a corresponding sub-image of the set of sub-images by providing the sub-image to a trained deep neural network; aggregating the outcome predictions of the set of outcome predictions to generate an aggregate outcome prediction; and providing the aggregate outcome prediction associated with the microscopy image.

Embodiment 18: The system of embodiment 17, wherein an outcome prediction of the set of outcome predictions corresponds to a prediction of disease progression within a future time period.

Embodiment 19: The system of embodiment 18, wherein the prediction of disease progression comprises metastasis of a tumor to a body region different from a body region associated with the test sample.

Embodiment 20: The system of any one of embodiments 17-19, wherein the microscopy image is a microscopy image that has not been physically stained.

Embodiment 21: The system of embodiment 20, wherein the microscopy image comprises a virtually stained microscopy image.

Embodiment 22: The system of embodiment 21, wherein the virtually stained microscopy image was generated by a trained machine learning model different from the trained deep neural network.

Embodiment 23: The system of any one of embodiments 17-22, wherein identifying the region of interest comprises filtering a background region based on an annotation of the region of interest.

Embodiment 24: The system of any one of embodiments 17-23, wherein the set of sub-images are selected with uniform probability from within the region of interest.

What is claimed is:

1. A method comprising:
receiving a microscopy image associated with a test sample;
identifying a region of interest of the microscopy image for analysis;
randomly selecting a set of sub-images from within the region of interest;
generating a set of outcome predictions, each outcome prediction associated with a corresponding sub-image of the set of sub-images by providing the sub-image to a trained deep neural network;
aggregating the outcome predictions of the set of outcome predictions to generate an aggregate outcome prediction; and
providing the aggregate outcome prediction associated with the microscopy image.

2. The method of claim 1, wherein an outcome prediction of the set of outcome predictions corresponds to a prediction of disease progression within a future time period.

3. The method of claim 2, wherein the prediction of disease progression comprises metastasis of a tumor to a body region different from a body region associated with the test sample.

4. The method of claim 1, wherein the microscopy image is a microscopy image that has not been physically stained.

5. The method of claim 4, wherein the microscopy image comprises a virtually stained microscopy image.

6. The method of claim 5, wherein the virtually stained microscopy image was generated by a trained machine learning model different from the trained deep neural network.

7. The method of claim 1, wherein identifying the region of interest comprises filtering a background region based on an annotation of the region of interest.

8. The method of claim 1, wherein the set of sub-images are selected with uniform probability from within the region of interest.

9. The method of claim 1, wherein the deep neural network comprises a convolutional neural network.

10. The method of claim 1, wherein the deep neural network comprises an attention mechanism configured to utilize a region of interest within a sub-image to generate a corresponding outcome prediction.

11. The method of claim 1, wherein the aggregate outcome prediction is a median outcome prediction of the set of outcome predictions.

12. A method comprising:
obtaining a set of microscopy images and corresponding ground truth predictions, each ground truth prediction indicating an outcome for a patient associated with the microscopy image;
dividing the set of microscopy images and corresponding ground truth predictions into a training set and a validation set;
performing an initial training of a deep neural network by:
providing sub-images from a region of interest of a given microscopy image from the training set to the deep neural network;
generating an aggregate outcome prediction for the given microscopy image based on outcome predictions associated with each sub-image of the given microscopy image; and
updating weights of the deep neural network based on a difference between the aggregate outcome prediction and the ground truth prediction for the given microscopy image; and
performing fine-tuning of the deep neural network using the validation set, wherein the fine-tuning comprises updating at least one hyperparameter.

13. The method of claim 12, wherein the at least one hyperparameter comprises a learning rate, a batch size, a weight decay, a learning scheduler, or any combination thereof.

14. The method of claim 12, wherein the fine-tuning of the deep neural network comprises providing sub-images from microscopy images included in the validation set to the initially-trained deep neural network.

15. The method of claim 12, wherein the microscopy image is a microscopy image that has not been physically stained.

16. The method of claim 15, wherein the microscopy image comprises a virtually stained microscopy image.

17. A system comprising:
one or more processors; and
one or more processor-readable media storing instructions which, when executed by one or more processors, cause performance of:
receiving a microscopy image associated with a test sample;
identifying a region of interest of the microscopy image for analysis;
randomly selecting a set of sub-images from within the region of interest;
generating a set of outcome predictions, each outcome prediction associated with a corresponding sub-image of the set of sub-images by providing the sub-image to a trained deep neural network;
aggregating the outcome predictions of the set of outcome predictions to generate an aggregate outcome prediction; and
providing the aggregate outcome prediction associated with the microscopy image.

18. The system of claim 17, wherein an outcome prediction of the set of outcome predictions corresponds to a prediction of disease progression within a future time period.

19. The system of claim 18, wherein the prediction of disease progression comprises metastasis of a tumor to a body region different from a body region associated with the test sample.

20. The system of claim 17, wherein the microscopy image is a microscopy image that has not been physically stained.

21. The system of claim 20, wherein the microscopy image comprises a virtually stained microscopy image.

22. The system of claim 21, wherein the virtually stained microscopy image was generated by a trained machine learning model different from the trained deep neural network.

23. The system of claim 17, wherein identifying the region of interest comprises filtering a background region based on an annotation of the region of interest.

24. The system of claim 17, wherein the set of sub-images are selected with uniform probability from within the region of interest.
